(19) United States
(12) Patent Application Publication
(10) Pub. No.: US 2005/0241651 A1
(43) Pub. Date: Nov. 3, 2005

(54) EXTERNAL INCONTINENCE CLAMP

(76) Inventor: HENRY RENNICH, CALGARY (CA)

Correspondence Address: STEPHEN J. LEWELLYN, 933 OLEANDER WAY SOUTH, SUITE 3, SOUTH PASADENA, FL, 33707, US

(21) Appl. No.: 11/161,011

(22) Filed: Jul. 19, 2005

Related U.S. Application Data
(63) Continuation-in-part of application No. 10/688,053, filed on Oct. 20, 2003.

(51) Int. Cl. A61F 5/48

(52) U.S. Cl. 128/885

(57) ABSTRACT

An external incontinence clamp for stopping involuntary voiding of urine is provided. The clamp includes a two opposed clamping members hingidly attached together at common ends with the opposite end of each member being releasable secured together at a spaced distance by a connection pin. The connection pin is easily released from the ends of the clamping members to allow voiding of urine. Shape conforming pads are provided and are attached to each clamping member. The pads are positioned to engage and partially conform to a penis to which the clamp is attached. The space between the first and second clamping members can be selectively increased or decreased to adjust the amount of pressure applied to the penis.

### CROSS-REFERENCE TO RELATED APPLICATION

This application claims the benefit of U.S. patent application Ser. No. 10/688,053, filed Oct. 20, 2003, the entirety of which is incorporated herein by reference.

### FIELD OF THE INVENTION

The present invention relates generally to male incontinency devices. More particularly, relating to an external incontinence device that is clamped to a male's penis to restrict urine flow through the urethra.

### DESCRIPTION OF THE PRIOR ART

Due to the anatomy of a penis, external devices that are clamped or other wise secured to a penis can be very effective in controlling urine flow through the urethra. The urethra is a passageway through which urine travels and it is located on the under side of the penis relatively close to the skin surface. A very small amount of pressure applied to the urethra will collapse the passageway and prevent urine from flowing through. There are many know prior art devices that attempt to accomplish this to control or prevent involuntary voiding of a bladder or to prevent urine leakage.

An example of an aforementioned device is disclosed in U.S. Pat. No. 4,942,866 to Timmons. This patent discloses an external incontinency clamp having two arcuate members hinged together at one end and a releasable fastener on the opposite ends, which are adapted to receive and clamp a penis therebetween.

Similarly, U.S. Pat. No. 3,147,754 to Koessler discloses a device for controlling incontinence having a U-shaped part with a cooperating cross bar, which together serve as a clamp for a penis.

Lastly, U.S. Pat. No. 5,571,125 to Chadwick discloses a penis-clamping device including a cushioned clamp that is hinged at one end and provided with an adjustable tensioning closure device at its other end.

While the above-described devices fulfill their respective, particular objectives and requirements, the aforementioned patents do not describe an external incontinence clamp for applying an equal force across a penis to prevent the penis from slipping towards either side of a centerline of the clamp, thereby preventing inadvertent opening of the urethra causing urine leakage.

Therefore, a need exists for a new and improved external incontinence clamp that can be used for preventing involuntary voiding of a bladder by restricting urine flow through the urethra. In this regard, the present invention substantially fulfills this need. In this respect, the external incontinence clamp according to the present invention substantially departs from the conventional concepts and designs of the prior art.

### SUMMARY OF THE INVENTION

In accordance with the present invention, an external incontinence clamp is provided.

In general, in one aspect the external incontinence clamp includes:
a first clamping member having a first end, a second end, and a mid portion extending between the first and second ends, the first end extending perpendicularly from the first clamping member in a direction inwardly of the clamp, the mid portion having an inward facing surface;
a rigid second clamping member having a first end, a second end, and a mid portion extending between the first and second ends, the first end extending perpendicularly from the second clamping member in a direction inwardly of the clamp, the mid portion having an inward facing surface;
a hinge pin, the first ends defining cooperating hinge pin receiving bores which are formed laterally through each end, the first ends are hingidly attached together by the hinge pin being releasable inserted into the hinge pin receiving bores allowing articulated movement of the first and second clamping members relative to one another; a first pad attached to the inward facing surface of the mid portion of the first clamping member; a second pad attached to the inward facing surface of the mid portion of the second clamping member; and
a connection pin, the connection pin being releasably received by the seconds to retain the second ends at a spaced distance.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

An object of the present invention is to provide an external incontinence clamp for stopping involuntary voiding of urine through a penis.

Still a further object of the present invention is to provide an external incontinence clamp that is easy to operate and does not require removal of the clamp for urination.

Lastly, it is an object of the present invention to provide an external incontinence clamp and method of operating the same that has a low cost of manufacture with regard to both materials and labor, and which accordingly is then susceptible of low prices of sale to the consuming public, thereby making such clamp economically available to the buying public.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is top plan view a preferred embodiment of the external incontinence clamp constructed in accordance with the principles of the present invention;

FIG. 2 is a side elevation view of the external incontinence clamp of FIG. 1 showing the positioning of the clamp on a penis;

FIG. 3 is a sectional view taken along line 3-3 in FIG. 1 illustrating a hinged coupling between clamping members;

FIG. 4 is a sectional view taken along line 4-4 in FIG. 1 illustrating a pinned coupling between clamping members;

FIG. 5 is a section view taken along line 5-5 in FIG. 2 illustrating a contact pad partially conforming to the shape of a penis;

FIG. 6 is the external incontinence clamp of FIG. 1 shown in an alternate positions illustrating adjustment of the distance between clamping members to change clamping force applied to the penis; and

FIG. 7 is the external incontinence clamp of FIG. 1 shown in alternate positions and being placed onto a penis for use.

The same reference numerals refer to the same parts throughout the various figures.

### DETAILED DESCRIPTION OF THE INVENTION

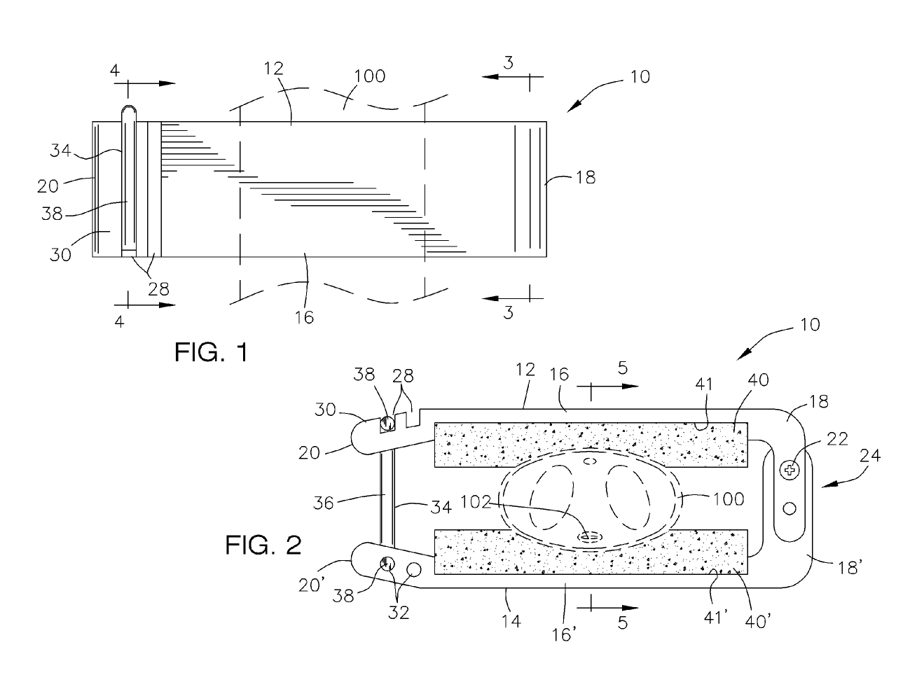

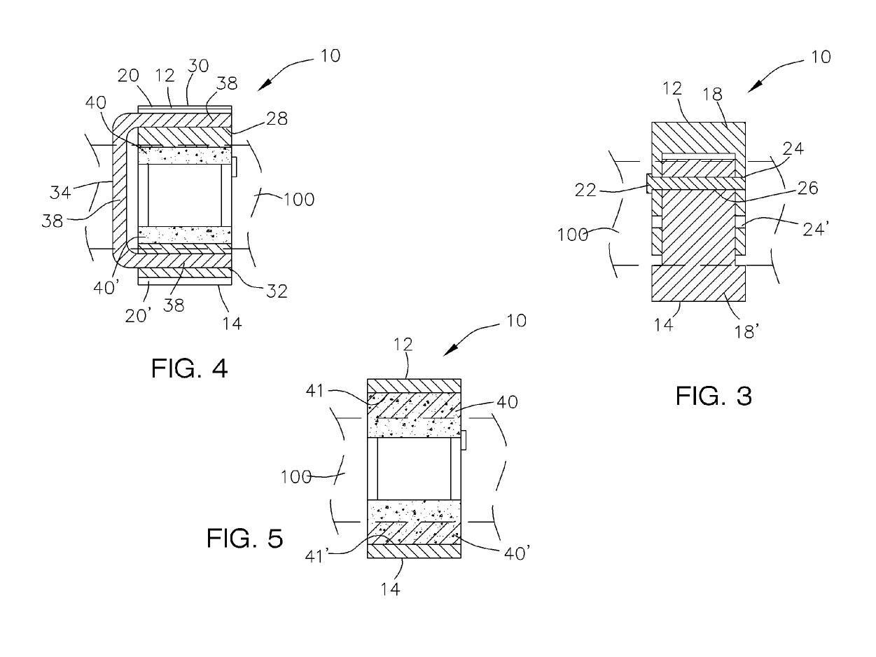

Referring now to the drawings, and particularly to FIGS. 1-6, a preferred embodiment of the external incontinence clamp of the present invention is shown and generally designated by the reference numeral 10.

As best illustrated in FIGS. 1 through 6, the external incontinence clamp 10 generally comprises an adjustable clamp 10 for preventing involuntary voiding of a bladder by restricting urine flow through the urethra of a penis. Through out the Figures, The clamp 10 is illustrated in use positioned on a penis 100, which is shown in broken line.

The clamp 10 includes a first clamping member 12 and an opposed second clamping member 14. The first and second clamping members 12 and 14 each include a first end 18 and 18′ respectively, a second end 20 and 20′ respectively and a mid portion 16 and 16′ respectively which is attached to and extends between the first and second ends.

The first clamping member 12 and the second clamping member 14 can be constructed from any material that affords rigidity to the members and is also hypoallergenic and non-irritating to the skin. Preferably, the first and second members 12 and 14 are constructed from Polyvinyl chloride (PVC).

The first end 18 of the first clamping member 12 is bent in a direction inwardly of the clamp 10 and the first end 18′ of the second clamping member 14 is bent in a direction inwardly of the clamp 10. One of the first ends, such as end 18 is bifurcated and receives the other first end 18′ between the furcations. Preferably, the first ends 18 and 18′ are bent inwardly such that each end forms a right angle with its respective clamping member. The first ends 18 and 18′ are pivotally secured to one another by a hinge pin 22 and form an adjustable hinged coupling 24 between the first clamping member 12 and the second clamping member 14.

The hinge pin 22 is received by cooperating hinge pin receiving bores 24 and 26 formed laterally through the first ends 18 and 18′ respectively. Preferably, the first ends 18 and 18′ include more than one set of cooperating hinge pin receiving bores 24 and 26, which permits adjustment of the distance between the first and second clamping members 12 and 14 approximate the first ends 18 and 18′.

For exemplary purposes only, the first end 18 of the first clamping member 12 is shown having a two hinge pin receiving bores 24 and 24′ with the hinge pin 22 received by cooperating hinge pin receiving bores 24 and 26. The distance between the first and second clamping members 12 and 14 can be adjusted by removing hinge pin 22 from bore 24 and aligning bore 24′ with bore 26 and then inserting hinge pin 22 through the now cooperating bores 24′ and 26. In this manner, the distance between the first and second clamping members 12 and 14 approximate the first ends 18 and 18′ is increased. It is to be understood that the first ends 18 and 18′ may having any number of sets and or configuration of cooperating hinge pin receiving bores, and that the total number of bores formed through each end 18 and 18′ can vary.

The second end 20 of the first clamping member 12 and the second end 20′ of the second clamping member 14 are each bent in a direction inwardly of the clamp 10. The second end 20 is bent inwardly such that an acute angle is made with reference line A, and the second end 20′ is bent inwardly such that an acute angle is made with reference line A′.

The second end 20 of the first clamping member 14 includes at least one slot 28 formed through an exterior surface 30 and along the width thereof (laterally across). The second end 20′ of the second clamping member 14 includes at least one connection pin receiving bore 32 formed therethrough in a direction along the width of the end 20′.

The second end 20 of the first clamping member 12 and the second end 20′ of the second clamping member 14 are releasably held together by a connection pin 34. The connection pin 34 includes a bridge portion 36 and two opposed legs 38 which extend outward and parallel to one another from the bridge portion. One opposed leg 38 is adapted to be received by the slot 28 of the second end 20 with the opposite leg adapted to be received by the connection pin receiving bore 32 of the second end 20′.

The dimension of the slot 28 is such that a tight interfittment is made between the slot and the leg 38 which is received by the slot. Additionally, the leg 38 which received by the bore 32 is rotatable within the bore along the axis of the leg, thereby allowing the entire connection pin 34 to rotate with respect to the clamp 10 about the leg and the second end 20′.

Preferably, at least two slots 28 are formed laterally across and through the exterior surface 30, and spaced longitudinally along the second end 20. Preferably at least two connection pin receiving bores 32 are formed at a spaced distance through the second end 20′. The inclusion of more than one slot 28 and more than one bore 32 permits adjustment of the distance between the first and second clamping members 12 and 14 approximate the second ends 20 and 20′. While it is preferred to include at least two slots 28 and two bores 32, it is contemplated that any number of slots and bores can be provided. For example, a plurality of slots 28 can be provided along with a single bore 32 or in the alternative a plurality of bores and a single slot can be provided.

The mid portion 16 of the first clamping member 12 includes a contact pad 40 and the mid portion 16′ of the second clamping member 14 includes an opposed contact pad 40′. The contact pads 40 and 40′ are attached to each mid portion 16 and 16′ along opposed facing surfaces 41 and 41′, such that the penis 100 is caused to be compressed between the contact pads when the clamp 10 is secured to the penis. Preferably, each contact pad 40 and 40′ is of a visto-elastic type foam which deforms slightly to the contour of the compressed penis 100 and acts to encapsulate the penis between the first and second clamping members 12 and 14. Most preferably, each contact pad is of a shape memory type foam, such as type manufactured by Zotefoams and sold under the name of EV50. The use of the shape memory foam allows the contact pads 40 and 40′ to repeatedly deform to the contour of the compressed penis 100 regardless of the prior or subsequent force exerted on the penis by the clamp 10.

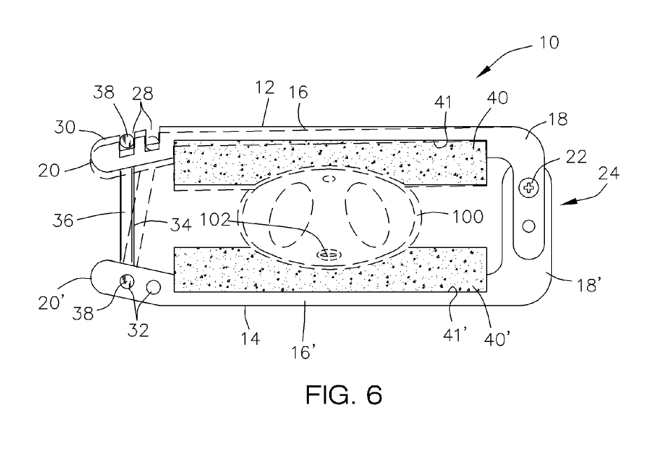

Now with reference to FIG. 6, a more detailed discussion of adjusting the distance between the first and second clamping members 12 and 14 at second ends 20 and 20′ will be add. It is to be understood this particular illustration of arrangement is only for exemplary purposes. More particularly, the clamp 10 is shown in solid line indicating a first positioning of the first and second clamping members 12 and 14, and the connection pin 34, and in broken line indicating a second positioning. The second positioning illustrates the first and second clamping members 12 and 14 having a smaller distance therebetween and thus in a position which exerts a greater force than that of the first positioning.

In the first position, it can be seen that a leg 38 of the connection pin 34 is positioned in to outward most bore 32 of the second end 20′ with the opposite leg 38 positioned in the outward most slot 28 of the second end 20. In this particular example, the positioning of the connection pin 34 allows for the greatest distance between the first and second clamping members 12 and 14, which in turns provide the least amount of force exerted by the clamp 10 on the penis 100.

In the second position, illustrated in broken line, it can be seen the leg 38 of the connection pin 34 received by the outward most slot 28 has been moved to the inward most slot and the opposed leg 38 remains in the outward most bore 32. In this positioning, the distance between the first and second clamping members 12 and 14 has been reduced approximate the second ends 20 and 20′, thereby applying a greater exerted force by the clamp 10 on the penis 100. In this particular example, the applied force can increased by moving the leg 38 from the outward most bore 32 into the inward most bore.

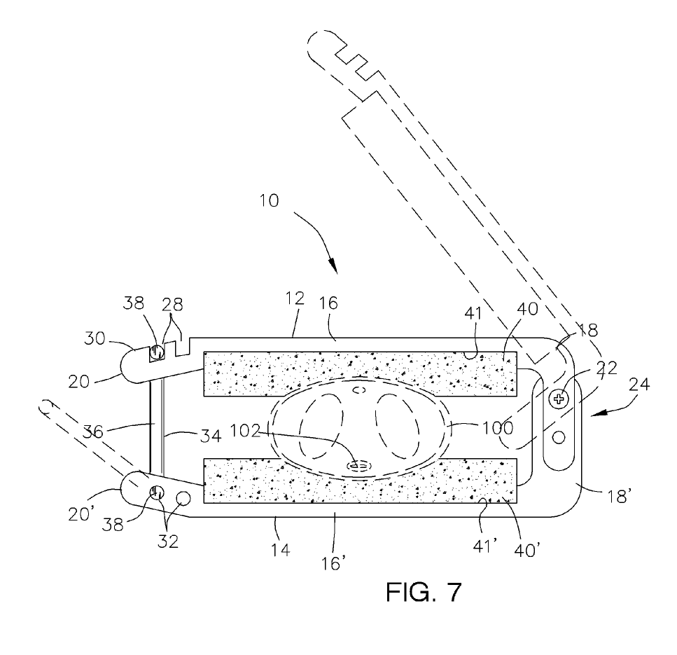

With reference to FIG. 7, in use it can know be understood, the clamp 10 is first opened and is positioned about the penis 100 such that the penis is aligned approximately about the mid point of the clamp. The clamp 10 is then closed about the penis 100 compressing the penis 100 between the contact pads 40 and 40′ and causing the urethra passage 102 to collapse. The clamp 10 is secured in position and clamped about the penis 100 by inserting one leg 38 of the connection pin 34 into a desired slot 28. If the user wishes to urinate, the second ends 20 and 20′ of the clamp 10 are slightly pushed together allowing the end 38 to be removed from the slot 28. The connection pin 34 is then rotated in a direction away from the end 20 permitting the first and second clamping members 12 and 14 to open about the penis 100 and allowing expansion of the urethra passage 102. After urination, the user simply reserves the above steps to resecure the clamp 10 about the penis 100. The user can adjust the positioning of the first ends 18 and 18′ and the location of the connection pin 34 as discussed in detail above to adjust the applied force on the penis 100 until the force applied is comfortable and is sufficient to prevent involuntary voiding.

While a preferred embodiment of the external incontinence clamp has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An external incontinence clamp for attachment to a penis comprising:
a rigid first clamping member having a first end, a second end, and a mid portion extending between said first and second ends, said first end extending perpendicularly from said first clamping member in a direction inwardly of the clamp, said mid portion having an inward facing surface;
a rigid second clamping member having a first end, a second end, and a mid portion extending between said first and second ends, said first end extending perpendicularly from said second clamping member in a direction inwardly of said clamp, said mid portion having an inward facing surface;
a hinge pin, said first ends defining cooperating hinge pin receiving bores which are formed laterally through each end, said first ends are hingidly attached together by said hinge pin being releasable inserted into said hinge pin receiving bores allowing articulated movement of said first and second clamping members relative to one another;
a first pad attached to said inward facing surface of said mid portion of said first clamping member;
a second pad attached to said inward facing surface of said mid portion of said second clamping member; and
a connection pin, said connection pin being releasably received by said seconds to retain said second ends at a spaced distance.

2. The external incontinence clamp of claim 1, wherein said first and second pads are of shape memory foam material.

3. The external incontinence clamp of claim 1, wherein:
said second end of said first clamping member is bent at an angle in a direction inwardly of the clamp, and defines a plurality of spaced slots formed latterly across and through an exterior surface thereof;
said second end of said second clamping member is bent at an angle in a direction inwardly of the clamp, and defines a plurality of spaced connection pin receiving bores formed laterally therethrough; and
said connection pin having a pair of opposed leg members, one leg member being releasably received by one of said lateral slots, and the oppose leg member being releasably received by one of said pin receiving bores.

4. The external incontinence clamp of claim 3, wherein one of said first ends defines a plurality of hinge receiving bores formed therethrough at spaced distances and longitudinally aligned.

5. The external incontinence clamp of claim 4, wherein one of said first ends is bifurcated and receives the other first end within the furcations of the bifurcated end.

6. The external incontinence clamp of claim 1, wherein one of said first ends defines a plurality of hinge pin receiving bores formed therethrough at spaced distances and longitudinally aligned.

7. The external incontinence clamp of claim 6, wherein one of said first ends is bifurcated and receives the other first end within the furcations of the bifurcated end.

8. The external incontinence clamp of claim 1, wherein said mid portions are substantially straight.

9. An external incontinence clamp for attachment to a penis comprising:
a rigid first clamping member having a first end, a second end, and a mid portion extending between said first and second ends, said first end extending perpendicularly from said first clamping member in a direction inwardly of the clamp, said mid portion having an inward facing surface;
a rigid second clamping member having a first end, a second end, and a mid portion extending between said first and second ends, said first end extending perpendicularly from said second clamping member in a direction inwardly of said clamp, said mid portion having an inward facing surface;
a hinge pin, said first ends defining cooperating hinge pin receiving bores which are formed laterally through each end, said first ends are hingidly attached together by said hinge pin being releasable inserted into said hinge pin receiving bores allowing articulated movement of said first and second clamping members relative to one another;
a first pad of shape memory foam material is attached to said inward facing surface of said mid portion of said first clamping member;
a second pad of shape memory foam material is attached to said inward facing surface of said mid portion of said second clamping member;
said second end of said first clamping member is bent at an angle in a direction inwardly of the clamp, and defines a plurality of spaced slots formed latterly across and through an exterior surface thereof;
said second end of said second clamping member is bent at an angle in a direction inwardly of the clamp, and defines a plurality of spaced connection pin receiving bores formed laterally therethrough; and
a connection pin; said connection pin having a pair of opposed leg members, one leg member being releasably received by one of said lateral slots, and the oppose leg member being releasably received by one of said pin receiving bores.

10. The external incontinence clamp of claim 9, wherein one of said first ends defines a plurality of hinge receiving bores formed therethrough at spaced distances in a longitudinal direction.

11. The external incontinence clamp of claim 10, wherein one of said first ends is bifurcated and receives the other first end within the furcations of the bifurcated end.

12. An external incontinence clamp for attachment to a penis comprising:
a rigid first clamping member having a first end, a second end, and a mid portion extending between said first and second ends, said first end extending perpendicularly from said first clamping member in a direction inwardly of the clamp, said mid portion having an inward facing surface;
a rigid second clamping member having a first end, a second end, and a mid portion extending between said first and second ends, said first end extending perpendicularly from said second clamping member in a direction inwardly of said clamp, said mid portion having an inward facing surface;
a first pad attached to said inward facing surface of said mid portion of said first clamping member;
an adjustable hinged coupling, said first ends being hingidly attached together by said adjustable hinged coupling;
a second pad attached to said inward facing surface of said mid portion of said second clamping member; and
a connection pin, said connection pin being releasably received by said seconds to retain said second ends at a spaced distance.

13. The external incontinence clamp of claim 12, wherein said first and second pads are of shape memory foam material.

14. The external incontinence clamp of claim 13, wherein said mid portions are substantially straight.

15. The external incontinence clamp of claim 12, wherein:
said second end of said first clamping member is bent at an angle in a direction inwardly of the clamp, and defines a plurality of spaced slots formed latterly across and through an exterior surface thereof;
said second end of said second clamping member is bent at an angle in a direction inwardly of the clamp, and defines a plurality of spaced connection pin receiving bores formed laterally therethrough; and
said connection pin having a pair of opposed leg members, one leg member being releasably received by one of said lateral slots, and the oppose leg member being releasably received by one of said pin receiving bores.

16. The external incontinence clamp of claim 15, wherein said first and second pads are of shape memory foam material.

17. The external incontinence clamp pf claim 16, wherein said mid portions are substantially straight.
